(19) United States
(12) Patent Application Publication
(10) Pub. No.: US 2020/0402659 A1
(43) Pub. Date: Dec. 24, 2020

(54) DISEASE SUFFERING PROBABILITY PREDICTION METHOD AND ELECTRONIC APPARATUS

(71) Applicants: Acer Incorporated, New Taipei City (TW); National Yang-Ming University, Taipei (TW)

(72) Inventors: Pei-Jung Chen, New Taipei City (TW); Tsung-Hsien Tsai, New Taipei City (TW); Liang-Kung Chen, Taipei (TW); Li-Ning Peng, Taipei (TW)

(73) Assignees: Acer Incorporated, New Taipei City (TW); National Yang-Ming University, Taipei (TW)

(21) Appl. No.: 16/667,930

(22) Filed: Oct. 30, 2019

(30) Foreign Application Priority Data
Jun. 19, 2019 (TW) 108121317

(51) Int. Cl. G16H 50/20; G06N 20/00; G16H 50/30; G16H 50/70

(52) U.S. Cl. CPC G16H 50/20; G16H 50/70; G16H 50/30; G06N 20/00

(57) ABSTRACT

A disease suffering probability prediction method and an electronic apparatus are provided. The method includes: determining a path length; obtaining a plurality of first paths conforming to the path length from a plurality of history data of a specific disease; obtaining a plurality of second paths positively related to the specific disease from the plurality of first paths; filtering the plurality of second paths to obtain a plurality of third paths, and establishing a prediction model according to the plurality of third paths; and inputting a path to be predicted to the prediction model and outputting a probability of suffering the specific disease.

### CROSS-REFERENCE TO RELATED APPLICATION

This application claims the priority benefit of Taiwan application serial no. 108121317, filed on Jun. 19, 2019. The entirety of the above-mentioned patent application is hereby incorporated by reference herein and made a part of this specification.

### BACKGROUND OF THE INVENTION

### 1. Field of the Invention

The present invention relates to a disease suffering probability prediction method and an electronic apparatus.

### 2. Description of Related Art

In general, based on the experience of diagnosis, doctors can determine what disease is likely to cause a specific disease. For example, dementia more likely occurs after diabetes. However, at present, it is mostly a single disease risk study, and there is no effective way to know which diseases are more likely to cause a specific disease sequentially.

### SUMMARY OF THE INVENTION

The present invention provides a disease suffering probability prediction method and an electronic apparatus, which can calculate information such as a proportion or probability of a specific disease suffered by a patient according to an order of diseases suffered by the patient.

The present invention provides a disease suffering probability prediction method, applied to an electronic apparatus. The method includes: determining a path length, the path length being a count of diseases; obtaining a plurality of first paths conforming to the path length from a plurality of history data of a specific disease according to the path length, the first path being composed of other diseases suffered sequentially before suffering the specific disease; obtaining a plurality of second paths positively related to the specific disease from the plurality of first paths according to the plurality of first paths; filtering the plurality of second paths to obtain a plurality of third paths, and establishing a prediction model according to the plurality of third paths; and inputting a path to be predicted to the prediction model and outputting a probability of suffering the specific disease for the path to be predicted, the path to be predicted being composed of a plurality of diseases.

The present invention provides an electronic apparatus, which includes: a storage circuit and a processor. The storage circuit records a plurality of modules. The processor accesses and executes the plurality of modules to perform the following operations: determining a path length, the path length being a count of diseases; obtaining a plurality of first paths conforming to the path length from a plurality of history data of a specific disease according to the path length, the first path being composed of other diseases suffered sequentially before suffering the specific disease; obtaining a plurality of second paths positively related to the specific disease from the plurality of first paths according to the plurality of first paths; filtering the plurality of second paths to obtain a plurality of third paths, and establishing a prediction model according to the plurality of third paths; and inputting a path to be predicted to the prediction model and outputting a probability of suffering the specific disease for the path to be predicted, the path to be predicted being composed of a plurality of diseases.

Based on the above, the disease suffering probability prediction method and the electronic apparatus of the present invention can find an order (also referred to as a path) of diseases suffered before suffering a specific disease (e.g., dementia) from history data. People with these paths will have a higher probability of suffering the foregoing specific disease in the future than those without them. In addition, the disease suffering probability prediction method of the present invention can further use the foregoing path to calculate information such as a proportion or probability of suffering a specific disease.

In order to make the aforementioned and other objectives and advantages of the present invention comprehensible, embodiments accompanied with figures are described in detail below.

### BRIEF DESCRIPTION OF THE DRAWINGS

FIG. 1 is a block diagram of an electronic apparatus in accordance with an embodiment of the present invention.

FIG. 2 is a flowchart of a method for filtering a path for predicting dementia using history data in accordance with an embodiment of the present invention.

FIG. 3 is a flowchart of a path filtering method in accordance with an embodiment of the present invention.

FIG. 4 is a flowchart of a multi-model variable filtering method in accordance with an embodiment of the present invention.

FIG. 5 is a flowchart of a disease suffering probability prediction method in accordance with an embodiment of the present invention.

### DESCRIPTION OF THE EMBODIMENTS

Reference will now be made in detail to the present preferred embodiments of the invention, examples of which are illustrated in the accompanying drawings. Wherever possible, the same reference numbers are used in the drawings and the description to refer to the same or like parts, components or steps.

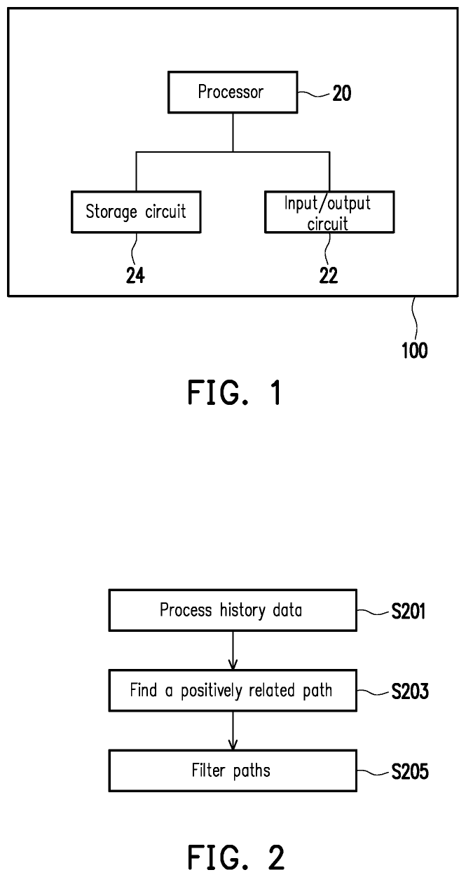

FIG. 1 is a block diagram of an electronic apparatus in accordance with an embodiment of the present invention.

Referring to FIG. 1, an electronic apparatus 100 includes a processor 20, an input/output circuit 22 and a storage circuit 24. The input/output circuit 22 and the storage circuit 24 are coupled to the processor 20 respectively. The electronic apparatus 100 is, for example, an electronic mobile apparatus such as a desktop computer, a server, a mobile phone, a tablet computer, or a notebook computer, which is not limited herein.

The processor 20 may be a central processing unit (CPU), or other programmable general-purpose or special-purpose microprocessors, digital signal processors (DSP), programmable controllers, application specific integrated circuits (ASIC), other similar components, or a combination of the aforementioned components.

The input/output circuit 22 is, for example, an input interface or circuit for obtaining related data from the outside of the electronic apparatus 100 or from other sources. In addition, the input/output circuit 22 may also transmit data generated by the electronic apparatus 100 to an output interface or circuit of another electronic apparatus, which is not limited herein.

The storage circuit 24 may be any type of fixed or mobile random access memory (RAM), read-only memory (ROM), flash memory or similar components, or a combination of the aforementioned components.

In the present exemplary embodiment, the storage circuit 24 of the electronic apparatus 100 stores a plurality of code segments, which are executed by the processor 20 after the code segments are installed. For example, the storage circuit 24 includes a plurality of modules by which respective operations of a disease suffering probability prediction method applied to the electronic apparatus 100 are respectively performed, where each module is composed of one or more code segments. However, the present invention is not limited thereto, and respective operations of the electronic apparatus 100 may be implemented by using other hardware forms.

It is to be noted that an order (also referred to as a path) of other diseases previously suffered by dementia patients may be related to future dementia before the diagnosis of dementia. The disease suffering probability prediction method of the present invention can find out the order of these diseases and provide information for doctors to assist in the prevention and treatment of dementia as a tool for assessing the risk of dementia. For example, a doctor can know which diseases are more susceptible to dementia sequentially, as well as the magnitude of a proportion or probability of dementia, etc.

In particular, the present invention is exemplified by dementia, but the present invention is not limited thereto. In other embodiments, the disease suffering probability prediction method of the present invention may also be applied to predict other diseases other than dementia, such as Parkinson's disease or other diseases, which are not limited herein. The following description is exemplified by dementia.

FIG. 2 is a flowchart of a method for filtering a path for predicting dementia using history data in accordance with an embodiment of the present invention. In the present exemplary embodiment, the definition of “path” is as follows: a combination of arranging several different diseases in an order, the order being an order conforming to time of the earliest occurrence of the diseases (or the earliest diagnosis). The definition of “path length” is as follows: a count of diseases in the foregoing path. In simple terms, the path may be diseases suffered (or diagnosed) by a patient sequentially, and the path length is a count of the diseases suffered by the patient.

Referring to FIG. 2, first, the processor 20 needs to perform a step of processing history data (step S201) to convert history data into data of the foregoing path.

In detail, the processor 20 needs to determine a path length according to requirements. Thereafter, the processor 20 may obtain a plurality of paths (hereinafter referred to as first paths) conforming to the path length from a plurality of history data of dementia according to the path length. In more detail, for each person in data, history data before suffering the dementia is taken, and then various diseases in the history are ranked at the time of the earliest occurrence (or time of the earliest diagnosis). From a disease ranking order of each person, all disease orders conforming to the foregoing path length are taken, and a relative disease order is maintained. All the paths that have appeared in the data are taken out.

The foregoing step of processing history data is exemplified below.

### Example of Step S201

It is assumed that the diagnosis history of patient A before dementia is: A->B->C->A->D->C. The foregoing A, B, C and D are diseases different from one other, and the diagnosis history may be a medical record of patient A. After obtaining the diagnosis history by the input/output circuit 22, the processor 20 may first rank the diseases according to time of the earliest diagnosis for each disease to obtain a disease ranking order: A->B->C->D. It is assumed that the previously determined path length is 3. The processor 20 may take out a combination of paths having a path length of 3 (i.e., composed of 3 diseases) from the foregoing disease ranking order and maintain a relative ranking between diseases, thereby obtaining the following 4 paths: A->B->C, A->B->D, A->C->D, and B->C->D.

It is assumed that all paths taken from the data of all patients (i.e., the foregoing first paths) are as follows: A->B->C, A->B->D, A->C->D, B->C->D, and B->A->C. These paths are taken as a feature, and the disease ranking order of patient A is converted into path data as shown in Table 1:

TABLE 1

| | A −> | A −> | A −> | B −> | B −> |
|---|---|---|---|---|---|
| | B −> C | B −> D | C −> D | C −> D | A −> C |
| Patient A | 1 | 1 | 1 | 1 | 0 |

Similarly, a path having a length of 3 may be obtained from data of each patient in the foregoing manner, and path data of each patient is recorded in Table 1 respectively.

Referring again to FIG. 2, next, the processor 20 performs a step of finding a positively related path (step S203). For example, the processor 20 may obtain a plurality of paths (also referred to as second paths) positively related to dementia from the first paths according to the foregoing plurality of first paths. In more detail, the processor 20 may calculate each path in the foregoing Table 1 separately using a machine learning method or a statistical method to determine whether a path is positively or negatively related to dementia. For example, the values of each column in the foregoing Table 1 are summed, paths summed to be greater than a threshold value are identified as positively related paths, and paths summed to be not greater than the threshold value are identified as negatively related paths. The processor 20 may preserve the positively related paths and delete the negatively related paths. In particular, a patient with a positively related path will have a higher chance of suffering dementia than a patient without a positively related path.

After performing step S203, the processor 20 performs a step of path filtering (step S205), thereby filtering the foregoing positively related paths (i.e., the second paths) to obtain a plurality of paths therefrom (also referred to as third paths). The processor 20 may establish a prediction model according to the third paths. In particular, this step mainly filters the foregoing second paths according to the prediction performance of dementia to find a third path with a better prediction ability from the second paths.

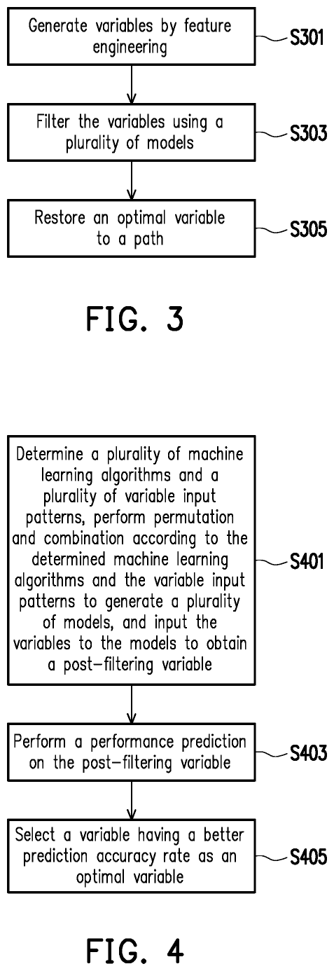

FIG. 3 is a flowchart of a path filtering method in accordance with an embodiment of the present invention.

Referring to FIG. 3, the foregoing step S205 may be further subdivided into steps S301 to S305. In more detail, in the process of performing step S205, the processor 20 generates a variable by feature engineering (step S301). For example, the processor 20 may generate a plurality of variables corresponding to a plurality of patterns according to the foregoing plurality of second paths.

In more detail, step S301 is to take out part or all of the diseases from each of the second paths and to generate a new variable accordingly. In particular, the generated new variable may have various different patterns by permutation and combination of a position of a disease, an order of diseases and a count of diseases.

Here, V(Count, Position, Order) is defined as the patterns of the new variable. “Count” represents a count of diseases taken from a path, which is equal to 1 minimally and equal to the length of the path maximally. “Position” represents a feature indicating whether a mode of taking a disease preserves a position of the disease in an original path, where the value thereof may be “Preserve Position (hereinafter referred to as PP)” or “Ignore Position (hereinafter referred to as IP)”. “Order” represents whether a mode of taking diseases preserves an order of diseases, where the value thereof may be is “Preserve Order (hereinafter referred to as PO)” or “Ignore Order (hereinafter referred to as IO)”. In particular, when the count of diseases is “1”, the order is meaningless, and a threshold of “Order” may be set to “X”.

A path length of 3 is taken as an example, where a new variable may have the following patterns (1) to (7).

| | |
|---|---|
| V(1, PP, X) | pattern (1) |
| V(1, IP, X) | pattern (2) |
| V(2, PP, PO) | pattern (3) |
| V(2, PP, IO) | pattern (4) |
| V(2, IP, PO) | pattern (5) |
| V(2, IP, IO) | pattern (6) |
| V(3, X, IO) | pattern (7) |

It is to be noted that in the case of a path, since the position is meaningless when the count is “3”, the value of the “Position” field is X, and when the “Order” field is “PO”, the original path is represented. Therefore, the pattern “V(3, X, PO)” is not recorded as a new variable.

In the present embodiment, the processor 20 performs the aforementioned feature engineering on all the second paths, obtains all new variables, and converts path data of each person into new variable data. In particular, in an embodiment, the processor 20 may pre-establish a comparison table for recording the correspondence between paths and variables. The processor 20 may generate a plurality of variables corresponding to the foregoing plurality of patterns according to the foregoing second paths and the comparison table.

The step of converting path data into new variable data is as follows: for a new variable X, if at least one path in the path data of a patient may generate X through the aforementioned process, in the new variable data of the patient, an X threshold is equal to 1, otherwise equal to 0.

The following is a path to illustrate the process of generating a new variable by feature engineering and the process of converting into path data.

### Example of Step S301

It is assumed that a path having a length of 3 is: A->B->C. Since the path length is 3, in step S301, a plurality of new variables is obtained from the foregoing patterns (1) to (7) by taking part or all of the diseases from 3 diseases (i.e., taking 1 disease, 3 diseases at most).

For the pattern V(1, PP, X), new variables may be obtained: A_1, B_2 and C_3. A number behind a baseline represents a position of a disease in the path.

For the pattern V(1, IP, X), new variables may be obtained: A, B and C.

For the pattern V(2, PP, PO), new variables may be obtained: A->B_1 and B->C_2. “->” represents an order. The variable “A->B_1” is taken as an example, where it represents that the order of suffering diseases is A->B, and A is at a first position in a path. For another example, the variable “B->C_2” represents that the order of suffering diseases is B->C, and B is at a second position in a path.

For the pattern V(2, PP, IO), new variables may be obtained: A&B_1&2, A&C_1&3 and B&C_2&3. The variable “A&B_1&2” is taken as an example, where it represents that diseases include A and B (in any order) and the two diseases are at first and second positions in a path. In other words, this example represents A->B or B->A from the first position in a path. For another example, the variable “A&C_1&3” represents that diseases include A and C (in any order) and the two diseases are at first and third positions in a path. In other words, the two positions (i.e., the first and third positions) may be A->C or C->A in a path. Other variables may be deduced by analogy, and the descriptions thereof are omitted herein.

For the pattern V(2, IP, PO), new variables may be obtained: A->B and B->C. The variable “A->B” is taken as an example, where it represents that diseases are sequentially A and B, but the positions of A and B in a path are not limited. For another example, the variable “B->C” represents that diseases are sequentially B and C, but the positions of B and C in a path are not limited. Other variables may be deduced by analogy, and the descriptions thereof are omitted herein.

For the pattern V(2, IP, TO), new variables may be obtained: A&B, A&C and B&C. The variable “A&B” is taken as an example, where it represents that diseases include A and B (in any order), and the positions of A and B in a path are not limited. For another example, the variable “A&C” represents that diseases include A and C (in any order), and the positions of A and C in a path are not limited. Other variables may be deduced by analogy, and the descriptions thereof are omitted herein.

For the pattern V(3, X, TO), a new variable may be obtained: A&B&C. The variable “A&B&C” is taken as an example, where it represents that diseases include A, B and C (in any order), and the positions of A, B and C in a path are not limited.

A total of 17 new variables may be obtained by the aforementioned patterns (1) to (7).

In an example, if there is a path A->B->C (threshold=1) in path data of patient B, the values of the aforementioned 17 new variables of patient B may be set to “1”. In another example, if A->B->C field=0 and A->B->G field=1 in path data of patient C, since the path A->B->G may also generate new variables such as A, B, A->B and A&B, the values of the new variables related to A and B may also be set to “1”.

Referring again to FIG. 3, after performing step S301, the processor 20 performs a step of filtering variables using a plurality of models (step S303). In more detail, the processor 20 may filter the plurality of variables generated in the foregoing step S301 using a plurality of models to obtain a plurality of optimal variables from the variables.

FIG. 4 is a flowchart of a multi-model variable filtering method in accordance with an embodiment of the present invention.

Referring to FIG. 4, the foregoing step S303 may be further subdivided into steps S401 to S405.

Referring to FIG. 4, in the step of filtering variables using a plurality of models, the processor 20 first determines a plurality of machine learning algorithms and a plurality of variable input patterns, and performs permutation and combination according to the determined machine learning algorithms and the variable input patterns to generate a plurality of models. Thereafter, the processor 20 may input the plurality of variables generated in the foregoing step S301 to the model generated in step S401 to obtain a post-filtering variable (step S401).

The variable input pattern refers to how variables are input to a machine learning model. A machine learning model (also referred to as One-model) may be established by inputting all variables once, and the variables are output using the model; or a plurality of models is established according to a variable pattern (also referred to as By-pattern) of an original variable, and a union operation is performed on output results of the plurality of models finally.

The following uses a random forest algorithm and a Logistic regression algorithm as an example. The processor 20 may generate a combination of the machine learning methods and the variable input patterns in Table 2 below:

TABLE 2

| Serial number of | Machine learning | Variable input | Count of |
|---|---|---|---|
| models | method | pattern | models |
| M01 | Random Forest | One-model | 1 |
| M02 | Random Forest | By-pattern | 7 |
| M03 | Logistic Regression | One-model | 1 |
| M04 | Logistic Regression | By-pattern | 7 |

In detail, in the step of generating a model in step S401, the processor 20 may establish a model (also referred to as a first model) for the aforementioned patterns (1) to (7), respectively, using a machine learning algorithm. Referring to Table 2, in the process of generating the first model, the processor 20 may establish models for the aforementioned patterns (1) to (7), respectively, using a random forest algorithm to generate seven models (i.e., model M02 in Table 2). For another example, the processor 20 may also establish models for the aforementioned patterns (1) to (7), respectively, using a Logistic regression algorithm to generate seven models (i.e., model M04 in Table 2).

In addition, in the step of generating a model in step S401, the processor 20 may generate a model (also referred to as a second model) corresponding to the aforementioned patterns (1) to (7) using a machine learning algorithm. Referring to Table 2, in the process of generating the second model, the processor 20 may establish a model (i.e., model M01 in Table 2) for the aforementioned patterns (1) to (7) using a random forest algorithm. In addition, the processor 20 may also establish models for the aforementioned patterns (1) to (7) using a Logistic regression algorithm to generate a model (i.e., model M03 in Table 2).

After obtaining the foregoing first model and second model, the processor 20 may input the plurality of variables obtained in step S301 to each of the foregoing first models (e.g., seven models in model M02 or seven models in model M04) to obtain post-filtering variables (also referred to as first post-filtering variables) output by each model. In other words, the seven models of model M02 is taken as an example, where there are seven groups of post-filtering variables. Thereafter, the processor 20 performs an union operation on the seven groups of post-filtering variables to obtain a group of post-filtering variables (also referred to as second post-filtering variables). Similarly, the seven models of model M04 is taken as an example, where there are seven groups of post-filtering variables. Thereafter, the processor 20 performs an union operation on the seven groups of post-filtering variables to obtain a group of post-filtering variables.

In addition, the processor 20 also inputs the plurality of variables obtained in step S301 to the foregoing second models to obtain post-filtering variables (also referred to as third post-filtering variables). For example, the model of model M01 is taken as an example, since model M01 includes only one model, there is only one group of post-filtering variables. Similarly, the model of model M03 is taken as an example, since model M03 includes only one model, there is only one group of post-filtering variables.

Thereafter, the processor 20 performs a performance prediction on the foregoing post-filtering variables (e.g., the second post-filtering variable and the third post-filtering variable) respectively using a plurality of third models (step S403) to select a variable having a better prediction accuracy rate therefrom as an optimal variable (step S405).

The foregoing models M01 to M04 are taken as an example, where four groups of post-filtering variables may be obtained after the foregoing step S401. In the step of performing the performance prediction on each group of post-filtering variables, the processor 20 establishes a plurality of models for each group of post-filtering variables using a plurality of machine learning methods, each model obtaining a prediction performance (e.g., prediction accuracy rate). These prediction performances are calculated statistically (e.g., averaging, maximizing, etc.) to obtain a performance representative of the group of post-filtering variables. Thereafter, in the process of selecting an optimal variable, the processor 20 compares the prediction performance of each group of post-filtering variables, and selects the best one (e.g., having the highest prediction performance) as an optimal variable.

Referring to FIG. 3 again, after performing step S303, the processor 20 restores the foregoing plurality of optimal variables to a path (also referred to as a third path) corresponding to the foregoing plurality of optimal variables (step S305). For example, the processor 20 restores the optimal variables to third paths corresponding to the optimal variables according to the comparison table used in the foregoing step S301.

The processor 20 may establish, after obtaining the third paths, a prediction model according to the third paths. Thereafter, when performing the risk assessment of dementia, the processor 20 may input a path to be predicted to the prediction model and output a probability of suffering a specific disease in the foregoing path to be predicted. The foregoing path to be predicted is composed of a plurality of diseases, and the count of the diseases is, for example, equal to the path length determined in the foregoing step S201.

An example of risk assessment for dementia may be as follows:

After obtaining the path to be predicted, the processor 20 uses, for example, historical data to calculate risk information such as a proportion of suffering dementia in the path to be predicted, and a proportion of the path to be predicted in a group of demented patients to all demented patients, and establishes a path and dementia risk information comparison table according to the foregoing information. Therefore, history data of any person is given and converted into path data, and then the risk of disease may be assessed using the comparison table. Table 3 below is a schematic diagram of a path and risk comparison table:

TABLE 3

| | | Total proportion |
|---|---|---|
| | Proportion of | of patients with |
| Path | dementia in path | dementia in path |
| Dizziness → anxiety → | 52% | 4.3% |
| dementia | | |
| Coronary heart disease → | 58% | 2.8% |
| stroke → dementia | | |

Another example of risk assessment for dementia may be as follows:

The processor 20 may establish prediction models for each group of post-filtering variables after the foregoing step S401, and take a prediction model of an optimal variable as an optimal model for generating a risk indicator such as a dementia probability or a prediction label. Thereafter, when the history of any person is given, the processor 20 may further convert a path into an optimal variable after converting into path data, and then use the optimal model to calculate a dementia risk indicator (e.g., probability or label).

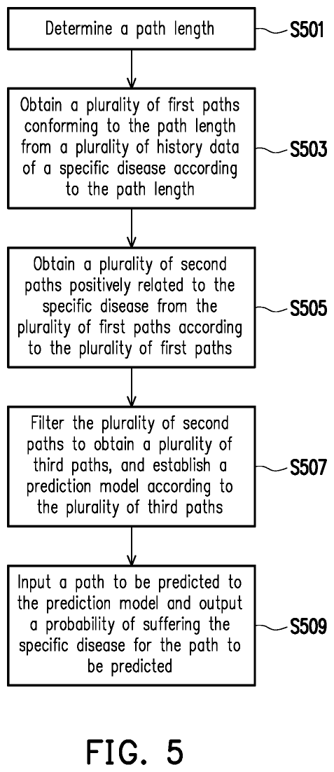

FIG. 5 is a flowchart of a disease suffering probability prediction method in accordance with an embodiment of the present invention.

Referring to FIG. 5, in step S501, a processor 20 determines a path length, where the path length is a count of diseases. In step S503, the processor 20 obtains a plurality of first paths conforming to the foregoing path length from a plurality of history data of a specific disease according to the foregoing path length, where the first path is composed of other diseases suffered sequentially before suffering the specific disease. In step S505, the processor 20 obtains a plurality of second paths positively related to the specific disease from the first paths according to the foregoing plurality of first paths. In step S507, the processor 20 filters the foregoing plurality of second paths to obtain a plurality of third paths, and establishes a prediction model according to the third paths. Finally, in step S509, the processor 20 inputs a path to be predicted to the prediction model and outputs a probability of suffering the specific disease for the path to be predicted, where the path to be predicted is composed of a plurality of diseases.

Based on the foregoing, the disease suffering probability prediction method and the electronic apparatus of the present invention can find an order (also referred to as a path) of diseases suffered before suffering a specific disease (e.g., dementia) from history data. People with these paths will have a higher probability of suffering the aforementioned specific disease in the future than those without them. In addition, the disease suffering probability prediction method of the present invention can further use the aforementioned path to calculate information such as a proportion or probability of suffering a specific disease.

Although the invention is described with reference to the above embodiments, the embodiments are not intended to limit the invention. A person of ordinary skill in the art may make variations and modifications without departing from the spirit and scope of the invention. Therefore, the protection scope of the invention should be subject to the appended claims.

What is claimed is:

1. A disease suffering probability prediction method, applied to an electronic apparatus, the method comprising:
determining a path length, the path length being a count of diseases;
obtaining a plurality of first paths conforming to the path length from a plurality of history data of a specific disease according to the path length, the first path being composed of other diseases suffered sequentially before suffering the specific disease;
obtaining a plurality of second paths positively related to the specific disease from the plurality of first paths according to the plurality of first paths;
filtering the plurality of second paths to obtain a plurality of third paths, and establishing a prediction model according to the plurality of third paths; and
inputting a path to be predicted to the prediction model and outputting a probability of suffering the specific disease for the path to be predicted, the path to be predicted being composed of a plurality of diseases.

2. The disease suffering probability prediction method according to claim 1, wherein the step of filtering the plurality of second paths to obtain the plurality of third paths comprises:
generating a plurality of variables corresponding to a plurality of patterns according to the plurality of second paths;
filtering the plurality of variables using a plurality of models to obtain a plurality of optimal variables from the plurality of variables; and
restoring the plurality of optimal variables to the plurality of third paths corresponding to the plurality of optimal variables.

3. The disease suffering probability prediction method according to claim 2, wherein the plurality of patterns are related to permutation and combination of a position of a disease, an order of diseases and a count of diseases in each of the plurality of second paths.

4. The disease suffering probability prediction method according to claim 2, wherein the step of filtering the plurality of variables using the plurality of models to obtain the plurality of optimal variables from the plurality of variables comprises:
determining a machine learning algorithm;
determining a plurality of variable input patterns; and
generating the plurality of models according to the determined machine learning algorithm and the plurality of variable input patterns.

5. The disease suffering probability prediction method according to claim 4, wherein the step of generating the plurality of models according to the determined machine learning algorithm and the plurality of variable input patterns comprises:
establishing a plurality of first models for the plurality of patterns respectively using the machine learning algorithm; and
establishing a second model for the plurality of patterns using the machine learning algorithm.

6. The disease suffering probability prediction method according to claim 5, wherein the step of filtering the plurality of variables using the plurality of models to obtain the plurality of optimal variables from the plurality of variables comprises:
inputting the plurality of variables to the plurality of first models to obtain a first post-filtering variable output by each first model in the plurality of models, and performing a union operation on the first post-filtering variable output by each first model in the plurality of models to obtain a second post-filtering variable;
inputting the plurality of variables to the second model to obtain a third post-filtering variable; and
performing a performance prediction on the second post-filtering variable and the third post-filtering variable respectively using a plurality of third models to select a variable having a better prediction accuracy rate from the second post-filtering variable and the third post-filtering variable as the plurality of optimal variables.

7. The disease suffering probability prediction method according to claim 4, wherein the machine learning algorithm comprises a random forest algorithm and a Logistic regression algorithm.

8. The disease suffering probability prediction method according to claim 2, wherein the step of generating the plurality of variables corresponding to the plurality of patterns according to the plurality of second paths comprises:
generating the plurality of variables corresponding to the plurality of patterns according to the plurality of second paths and a comparison table.

9. The disease suffering probability prediction method according to claim 7, wherein the step of restoring the plurality of optimal variables to the plurality of third paths corresponding to the plurality of optimal variables comprises:
restoring the plurality of optimal variables to the plurality of third paths corresponding to the plurality of optimal variables according to the comparison table.

10. An electronic apparatus, comprising:
a storage circuit, recording a plurality of modules; and
a processor, accessing and executing the plurality of modules to perform the following operations:
determining a path length, the path length being a count of diseases;
obtaining a plurality of first paths conforming to the path length from a plurality of history data of a specific disease according to the path length, the first path being composed of other diseases suffered sequentially before suffering the specific disease;
obtaining a plurality of second paths positively related to the specific disease from the plurality of first paths according to the plurality of first paths;
filtering the plurality of second paths to obtain a plurality of third paths, and establishing a prediction model according to the plurality of third paths; and
inputting a path to be predicted to the prediction model and outputting a probability of suffering the specific disease for the path to be predicted, the path to be predicted being composed of a plurality of diseases.
